(19) United States
(12) Patent Application Publication
(10) Pub. No.: US 2020/0010794 A1
(43) Pub. Date: Jan. 9, 2020

(54) METHOD OF INDUCING SPORULATION IN BACILLUS COAGULANS

(71) Applicant: Symbiosis International Deemed University, Maharashtra (IN)

(72) Inventor: Vinay RALE, Maharashtra (IN)

(21) Appl. No.: 16/490,947

(22) PCT Filed: Mar. 8, 2018
(86) PCT No.: PCT/IB2018/051509

§ 371 Date: Sep. 4, 2019

(30) Foreign Application Priority Data
Mar. 8, 2017 (IN) 201721008146

(51) Int. Cl. C12N 3/00; C12N 1/20

(52) U.S. Cl. CPC C12N 3/00; C12N 2500/05; C12N 2500/76; C12N 1/20

(57) ABSTRACT

The invention relates to methods for inducing sporulation in Bacillus coagulans strains wherein excessive sporulation is induced by presence of certain nutrients and minerals up to a level of 109 spores/ml. The spores are tadpole-like endospores and the method discloses specific parameters of growth medium and incubation conditions to be used to induce sporulation in Bacillus coagulans strains.

### TECHNICAL FIELD

The present subject matter described herein, in general, relates to the field of probiotics, and more particularly, to a method of inducing sporulation in probiotic lactic acid bacterium Bacillus coagulans.

### BACKGROUND

Probiotics are live microbial culture supplements. The consumption of probiotics by humans and animals result in improvement in intestinal microbial (microbiome) balance, improved nutrition and growth and prevention of gastrointestinal disorders. The usage of probiotics can be in two ways, namely, prophylactic and therapeutic.

Bacillus coagulans, previously known as Lactobacillus sporogenes, is a probiotic supplement that is capable of surviving stomach acid to reach the small intestine. Bacillus coagulans is unique among probiotics in that it naturally possesses a protective coating making it resistant to extreme temperatures and the acidity of the stomach and bile acids. These characteristics give Bacillus coagulans a greater shelf-life stability and a natural advantage for survivability in the intestines. Like all probiotics, Bacillus coagulans is a transient colonizing probiotic because it does not typically inhabit the human gastrointestinal tract. Amongst the Spore-Forming Lactic Acid Bacteria (SFLABs), the most prominent is Bacillus coagulans and is the probiotic of choice in the pharmaceutical world.

Based upon a survey conducted, it has been observed that several strains of Bacillus coagulans show considerable variation in sporulation when grown on peptone containing media deficient in minerals which are essential for achieving maximum sporulation. Therefore, many strains fail to produce spores. Thus, there is a long-standing need for an improved method or process that facilitates in producing adequate endospores in the Bacillus coagulans on an economical medium scalable to commercial levels.

### SUMMARY

Before the present method or process and components/apparatuses implementing the method/process is described, it is to be understood that this disclosure is not limited to the particular method/process/apparatus and its arrangement as described, as there can be multiple possible embodiments which are not expressly illustrated in the present disclosure but may still be practicable within the scope of the disclosure as determined by claims. It is also to be understood that the terminology used in the description is for the purpose of describing the particular versions or embodiments only and is not intended to limit the scope of the present subject matter. This summary is not intended to identify essential features of the subject matter nor it is intended for use in detecting or limiting the scope of the subject matter.

In accordance with aspects of the present disclosure, a method for producing endospores (‘tadpole’-like) exceeding 10⁹/ml in Bacillus coagulans is described herein. In order to facilitate sporulation in Bacillus coagulans, the present disclosure proposes employing various media ingredients including minerals and supplements at optimal environmental conditions so that the endospores (‘tadpole’-like) exceed 10⁹/ml. In accordance with aspects of the present disclosure, in order to obtain the desired media ingredients and the optimal conditions, several permutations and combinations of minerals and environmental conditions are considered by way of carrying out extensive experiments on the strains of the Bacillus coagulans until optimal conditions are achieved that facilitate in obtaining spores of Bacillus coagulans in excess of 10⁹/ml.

### DETAILED DESCRIPTION

The foregoing detailed description of embodiments is better understood when read in conjunction with the appended drawings. For the purpose of illustrating the disclosure, there is shown in the present document example constructions of the disclosure; however, the disclosure is not limited to the specific design disclosed in the document and the drawings.

The detailed description is described with reference to the accompanying figures. In the figures, the left-most digit(s) of a reference number identifies the figure in which the reference number first appears. The same numbers are used throughout the drawings to refer like features and components.

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. It must also be noted that, the singular forms “a,” “an,” and “the” include plural references unless the context clearly dictates otherwise.

The exemplary embodiments described herein and claimed hereafter may be suitably practiced in the absence of any recited feature, element or step that is, or is not, specifically disclosed herein. For instance, references in this written description to “one embodiment,” “an embodiment,” “an example embodiment,” and the like, indicate that the embodiment described can include a particular feature, structure, or characteristic, but every embodiment may not necessarily include the particular feature, structure, or characteristic. The disclosed embodiments are merely exemplary of various forms or combinations. Moreover, such phrases are not necessarily referring to some embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described.

No or terminology in this application should be construed as indicating any non-claimed element as essential or critical. The use of any and all examples, or example language (e.g., “such as”) provided herein, is intended merely to better illuminate example embodiments and does not pose a limitation on the scope of the claims appended hereto unless otherwise claimed.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller subranges are also included. The upper and lower limits of these smaller ranges are also included therein, subject to any specifically excluded limit in the stated range. For example, a range of “about 0.1% to about 5%” or “about 0.1% to 5%” may be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The present invention relates to methods and processes involving use of method for producing sporulation in a strain of microorganisms belonging to Bacillus genus. The present invention contains biological material which are commonly found throughout the territory of India as matter of common knowledge and the source of the extracts used have been indicated in the application. The presently characterized strain of the microorganism belonging to Bacillus genus was obtained from Advanced Enzyme Technologies Ltd, Sinnar, Nashik, Maharashtra and is commercially available to everybody. All the requisite formalities related to the present application under the Biodiversity Act 2002, if any, will be undertaken for compliance before the grant of the patent.

It has been widely accepted and realized that amongst the Spore-Forming Lactic Acid Bacteria (SFLABs), the most prominent is Bacillus coagulans and therefore is the probiotic of choice in the pharmaceutical world. However, in the existing scenario, it is observed that several strains of Bacillus coagulans exhibit considerable variation in sporulation when grown on peptone. This is primarily due to deficiency of minerals and supplements that are essential for obtaining maximum sporulation. Therefore, the present disclosure proposes a simple method or process for obtaining spores of Bacillus coagulans. More specifically, the present disclosure proposes a method/process of inducing sporulation in Bacillus coagulans that results in producing more than 10⁹/ml endospores (‘tadpole’-like).

In accordance with an embodiment of the present disclosure, in order to induce sporulation in Bacillus coagulans, several wet laboratory-based experiments involving many available strains were carried by employing different permutations and combinations of media ingredients and environmental conditions.

In one embodiment, the minerals particularly include manganese in the salt supplement and supplements include tomato juice, malic acid and the like.

In an exemplary embodiment, juice from tomato may be used as an essential ingredient of the growth medium. Briefly, ripe tomatoes may be steamed, pulped in a blender and juice filtered out. The juice may be sterilized separately at 105 degrees C. (5 pound/square inch) for 10 min and may be incorporated aseptically into the basal sterilized medium.

In an exemplary embodiment, the invention relates to a method for inducing sporulation in a strain of microorganism belonging to Bacillus genus, said method comprising a first step of addition of a predetermined amount of inoculum of said strain of microorganism belonging to Bacillus genus in a growth medium comprising at least a predetermined amount of yeast extract, a predetermined amount of skim milk powder, a predetermined amount of manganese sulphate, a predetermined amount of juice obtained from tomato, trace amount of salts belonging to nickel and cobalt and distilled water, a second step of incubating a strain of microorganism belonging to Bacillus genus in said growth medium of step one for a time period in the range of 24 hours to 48 hours, at a temperature in the range of 30 degree Celsius to 40 degrees Celsius and at a pH of 6.0, and wherein said growth medium comprises a dissolved oxygen content of at least 90%, and a third step of harvesting spores of said strain of microorganism belonging to Bacillus coagulans species at the end of said second step.

In another exemplary embodiment, the environmental conditions on said strains of Bacillus coagulans may include alkaline pH and a temperature of 30° C., etc. In one embodiment, the several experiments involved may either be carried out under stationary conditions or stirred conditions. In one embodiment, the experiments under stirred conditions may be carried out using an environmentally controlled shaker and a fermenter (e.g., New Brunswick Fermenter). Further, the volumes of various media for the experiments may range from a bench-scale (e.g. within a range of 50 ml-1 liter carried out on the shaker) to a pilot-scale (e.g. within a range of 2 liters to 5 liters carried out in the fermenter).

In accordance with an embodiment of the present disclosure, based upon the several experiments conducted with different permutations and combinations of media ingredients and supplements along with the environmental conditions, optimal conditions of alkaline pH and a temperature of 30° C. may be achieved that results in obtaining more than 10⁹/ml spores of Bacillus coagulans. Spore counts may be obtained using Neubaur Cell Counting Chamber using high power magnification (400×) on Compound Microscope. For clarity slides may be examined under Phase Contrast Microscope. The spores for final use may be ‘harvested’ by high speed centrifugation, washed with buffer and recentrifuged to obtain a concentrated volume which may be then freeze-dried in an industrial process.

In one embodiment, a method of inducing sporulation in Bacillus coagulans was implemented in accordance with the optimal conditions as described above. In this embodiment, one or more specific milk-based ingredients were included in a basal medium containing a strain of Bacillus coagulans. Further, in this embodiment, change in growth and multiplication of Bacillus coagulans in the selected strain was observed. Furthermore, in this embodiment, a spore concentration exceeding 10⁹/ml in a time span of 24-48 hours in said Bacillus coagulans was obtained. It must be noted that the said spore concentration (exceeding 10⁹/ml in a time span of 24-48 hours) was obtained without having to resort to any physical or chemical intervention in the said method.

Exemplary embodiments discussed above may provide certain advantages. Some embodiments of the present disclosure may enable a fermentation process for sporulation in Bacillus coagulans which is based on easily available, lost-cost media ingredients and combined with minimum process-cycle time, among several other requirements.

In another exemplary embodiment, the process may comprise a first step of sterilizing specified medium in a predetermined sized fermenter, may comprise a second step of addition of predetermined volume of inoculum, may further comprise a third step of switching on the fermenter at specified conditions and monitoring periodically the pH status and sporulation progression through microscopy.

Some embodiments of the present disclosure may enable a monitoring course of progression of the said process of sporulation in Bacillus coagulans by simple observations at production volumes. The monitoring of the progression of the said method/process may assist in making the process/method more robust with minimal interventions, that is the process may not use any physical or chemical interventions including various temperature changes and/or changes in pH and/or chemical additives to induce sporulation.

The embodiments, examples and alternatives of the preceding paragraphs, the description, including any of their various aspects or respective individual features, may be taken independently or in any combination. Features described in connection with one embodiment are applicable to all embodiments, unless such features are incompatible.

The present disclosure can be embodied in many other forms or carried out in other ways, without departing from the spirit or essential characteristics thereof, and the above-mentioned embodiment of the disclosure have been disclosed in detail only for illustrative purposes. It is understood that the disclosure is not limited thereto but is susceptible of numerous changes and modifications as known to those skilled in the art, and all such variations or modifications of the disclosed system, including the rearrangement of parts, lie within the scope of the present disclosure.

The foregoing description shall be interpreted as illustrative and not in any limiting sense. A person of ordinary skill in the art would understand that certain modifications could come within the scope of this disclosure.

Although implementations for have been described in language specific to features and/or processes, it is to be understood that the disclosure is not necessarily limited to the specific features or processes described. Rather, the specific features and processes are disclosed as examples of implementations for inducing sporulation in Bacillus coagulans.

### Example: Inducing Sporulation of Bacillus coagulans

| | |
|---|---|
| Parameter | Value |

Growth Medium

| Growth Medium constituents | | |
|---|---|---|
| Yeast Extract | 8-10 | g/L |
| Skim Milk powder | 40 | g/L |
| Tomato juice | 5-10% (volume/volume) | |
| Manganese Sulphate | 1 | mg/L |

| | |
|---|---|
| Nickel | Trace |
| Cobalt | Trace |
| Distilled water | Depending on batch volume |

Inoculum

| | |
|---|---|
| Bacillus coagulans st. | 2% (volume/volume) |

Incubation Conditions

| | |
|---|---|
| Temperature | 30-40 degrees Celsuis |
| Dissolved Oxygen Content | >90% |
| Incubation time | 24 hours-48 hours |
| pH | 6.0 |

Result

| | |
|---|---|
| Sporulation | >10⁹ spores/ml |

The foregoing description shall be interpreted as illustrative and not in any limiting sense. A person of ordinary skill in the art would understand that certain modifications could come within the scope of this disclosure.

1. A method for inducing sporulation in a strain of microorganism belonging to Bacillus genus, said method comprising:
a. a first step of addition of a predetermined amount of inoculum of said strain of microorganism belonging to Bacillus genus in a growth medium comprising at least a predetermined amount of yeast extract, a predetermined amount of skim milk powder, a predetermined amount of manganese sulphate, a predetermined amount of juice obtained from tomato, and a trace amount of salts belonging to nickel and cobalt and distilled water,
b. a second step of incubating a strain of microorganism belonging to Bacillus genus in said growth medium of step one for a time period in the range of 24 hours to 48 hours, at a temperature in the range of 30 degree Celsius to 40 degrees Celsius and at a pH of 6.0, and wherein said growth medium comprises a dissolved oxygen content of at least 90%, and
c. a third step of harvesting spores of said strain of microorganism belonging to Bacillus coagulans species at the end of said second step.

2. The method of claim 1, wherein said strain of microorganism belonging to Bacillus genus is a strain belonging to the Bacillus coagulans.

3. The method of claim 2, wherein said sporulation of said strain of microorganism belonging to Bacillus coagulans species is at an amount of at least 10⁹ spores/millilitre of growth medium.

4. The method of claim 3, wherein said sporulation comprises tadpole-like endospores.

5. The method of claim 1, wherein inoculum is added at an of amount of 2% v/v.

6. The method of claim 1, wherein said predetermined amount of yeast extract comprises 8-10 g/l, and wherein said predetermined amount of skim milk powder comprises up to 40 g/l, said predetermined amount of manganese sulphate comprises up to 1 mg/l, and said predetermined amount of juice obtained from tomato comprises an amount of at least 5-10% volume/volume.

7. The method of claim 1, wherein said method is carried out in a lab scale or pilot scale fermenter having a capacity of 5 litres or more.

8. The method of claim 1, wherein said method does not comprise any physical or chemical intervention.

9. A composition for a growth medium for inducing sporulation in a strain of microorganism belonging to Bacillus coagulans sp., said composition comprising at least a predetermined amount of yeast extract, a predetermined amount of skim milk powder, a predetermined amount of manganese sulphate, a predetermined amount of juice obtained from tomato, trace amount of salts belonging to nickel and cobalt and distilled water.

10. The composition of claim 9, wherein said predetermined amount of yeast extract comprises 8-10 g/l, said predetermined amount of skim milk powder comprises up to 40 g/l, said predetermined amount of manganese sulphate comprises up to 1 mg/l, and said predetermined amount of juice obtained from tomato comprises an amount of at least 5-10% volume/volume.
